(19) United States
(12) Patent Application Publication
(10) Pub. No.: US 2018/0252629 A1
(43) Pub. Date: Sep. 6, 2018

(54) SUBSTRATE PERMEABILITY MEASURING DEVICE

(71) Applicant: HZ-DR. HANS JÜRGEN HAHN UND DR. THOMAS ZUMBROICH GBR, Bonn (DE)

(72) Inventors: Thomas ZUMBROICH, Bonn (DE); Hans Jürgen HAHN, Landau (DE)

(21) Appl. No.: 15/769,656

(22) PCT Filed: Oct. 18, 2016
(86) PCT No.: PCT/EP2016/074917

§ 371 Date: Apr. 19, 2018

(30) Foreign Application Priority Data
Oct. 19, 2015 (DE) 20 2015 105 525.0
Oct. 19, 2015 (EP) 15190408.3

(51) Int. Cl. G01N 15/08; G01N 33/24

(52) U.S. Cl. CPC G01N 15/0826; G01N 33/24

(57) ABSTRACT

An underground permeability measuring apparatus includes an injection member formed as a lance to be introduced into the underground, having a cavity for guiding a fluid to at least one exit port, which preferably is arranged in the free end region of the injection member, through the injection member and the exit port into the underground surrounding the injection member is injectable. An injection member for introduction into an underground includes a cavity for guiding a fluid to at least one exit port, which preferably is arranged in the free end region of the injection member.

The apparatus further includes a pressure-increasing device, through which the fluid is injectable by overpressure through the injection member and the exit port into the underground surrounding the injection member.

### TECHNICAL FIELD

The disclosure relates to an underground permeability measuring apparatus and a method for permeability measurement of soils, especially the colmation in undergrounds of waters.

### BACKGROUND

Permeability or infiltration ability of undergrounds, respectively, is, among others, significantly affected by colmation. Colmation refers to the clogging of the fine gap system in the sands, gravels and grits of the riverbeds and reduction of water flow in the sediments.

In Europe, there is an every year erosion of soil of 970 million tons. The major part of those fine sediments is introduced into the flowing waters. Moreover, particle-size pollutants of the organic or inorganic type will be introduced into creeks and rivers through discharges from the sewer system in the events of heavy rain falls in areas of settlement and across large impervious surfaces (such as e.g. motorways). This also results in colmation.

Said pollutants will clog the fine gap systems in the sands, gravels and grits of the riverbeds, the so-called hyporheic zone. Thus, flow and exchange of water between surface water, hyporheic zone and ground water is suppressed.

Colmation, inter alia, also is of concern in biological sewage plants and soakaways Moreover, soil pores may become clogged (colmated) by the root growth. Another phenomenon resides in silting of the top soil by heavy or continual rainfalls, which poses a problem in agriculture.

When examining and evaluating colmated flowing waters, the hyporheic zone mostly is completely omitted, which mostly results in diffuse outcome. Even though the structure and water quality are rated good or very good, ecological evaluation reveals that the condition is only mediocre (“general degradation”). As colmation occurs in all running waters that flow through agricultural regions and areas of settlement, the major part of European creeks and rivers will probably be affected therefrom.

However, for the time being, there are no standard methods for identifying and evaluating colmation of running water sediments. As far as taken into account at all, colmation is being roughly estimated. So far, methods for the quantitative identification of colmation do not exist.

Measuring apparatuses and methods are known which allow identification of permeability of undergrounds; however, they are not suitable for identification of the inner colmation. For example, FR 2 576415 describes a measuring apparatus and method for the identification of underground permeability, wherein a measuring apparatus is introduced in a pre-drilled borehole. Described is a pressure pulsation measurement in terrestrial underground. Finally, the method is elaborate and cost intensive. In U.S. Pat. No. 5,548,991, an injection apparatus is described, which also allows identification of soil permeability. An injection member is introduced into the underground with the help of lubricant. Following removal of the injection member, the borehole will be sealed with a sealant. Both the lubricant and the sealant will change the underground, such that measurement of the inner colmation is not possible any more.

The approaches generally used so far for identifying the inner colmation, may be divided into qualitative methods, metrological methods and calculation methods. Among the qualitative methods there are, for example, visual evaluation, the so-called boot test, and the optical evaluation on dry portions of the sole. All of these qualitative methods suffer from the disadvantage that they are very subjective, and in this respect do not allow any objective comparison of different evaluation methods.

The metrological methods include sediment traps, sieve analyses, groundwater level measurements and runoff measurements. Sediment traps only allow qualitative evaluation, which in turn depends on the experimental setup. Sieve analyses are elaborate, and graduated evaluation of the colmation hardly is possible. Evaluation of the ground water level does not allow differentiated evaluation of the colmation either, whereas in runoff measurements, evaluating of the colmation is not unambiguous.

Calculation methods are based on the permeability as a function of time. Excessive effort is to be made and measurement of many input parameters is required. One methodology for elicitation of the inner under water colmation includes introduction of a round steel pin of 33 cm in length into the soil or the gravel sole or gravel bed, respectively. The steel pin projects into the soil by 16.5 cm. A cord string forming a loop is laid around the pin. A spring balance is attached at the other end of the cord string. While force is being increased, pulling is done perpendicularly to the pin until the pin is being loosened out of the gravel. The force required may be read on the spring balance. What is of disadvantage with this method is, among others, that the resistance depends on non-visible situations in the soil. Moreover, it has been proven that numerous measurements are required, in order to gain a relatively objective result.

The infiltration ability of the underground is also of importance in building site investigations or in permeability measurements in ground water conductors as well as soils. Beyond said methods, also for this application field, no measuring apparatuses or easy-to-use methods are known for terrestrial soils. For example, in the manufacture of infiltration wells, infiltration ditches or retention soil filter basins, clear identification of infiltration ability of the soil or the underground is required in advance. It is not before rainwater may infiltrate at sufficient rate, that such a technical French drain plant is approvable.

### SUMMARY

The present disclosure provides an underground permeability measuring apparatus, by which the degree of the inner colmation may safely and reproducibly be measured. Furthermore, for example, examination of building ground in view of the infiltration ability of the underground as well as permeability measurements in ground water conductors and soils is also to be possible. The underground permeability measuring apparatus is to be of robust and simple construction, possible error sources are to be minimized. Moreover, the manufacture of the underground permeability measuring apparatuses is to be of low cost and the maintenance and upkeep are to be performable with low effort. Furthermore, the present disclosure provides a method for measuring the inner colmation, which especially may be performed with the underground permeability measuring apparatus according to the disclosure.

According to the disclosure, the advantages will be realized by providing an underground permeability measuring apparatus having an injection member formed as a lance to be introduced into the underground, having a cavity for guiding a fluid to at least one exit port, which is arranged in the free end region of the injection member, through the injection member and the exit port into the underground surrounding the injection member is injectable.

Furthermore, the advantages will be realized by providing a method for measuring colmation in undergrounds of waters including the process steps introducing into an underground an injection member formed as a lance and having a cavity for guiding fluid to at least one exit port, which exit port is arranged in the free end region of the injection member, injecting fluid by way of overpressure through an injection member and the exit port into the underground, while setting and maintaining two parameters, selected from the group consisting of: fluid volume, injected; timing of fluid injection; and pressure level of fluid injection; and identifying one of the said parameters which has not been set and maintained.

The term underground, in the sense of the disclosure, includes all undergrounds, especially loose sediments (creek sediments), soils, debris and other geological undergrounds.

The term fluid includes suitable gases and liquids. In the following, the disclosure will be explained by the use of liquids as a fluid, but the disclosure shall not be limited to use of a liquid.

The inventors have revealed that a resilient method for the quantitative measurement of the colmation must be able to identify the permeability of the sediments for water. The result must yield some type of parameter, which is comparable to the auxiliary permeability value for the ground water conductor (Kf value).

According to the disclosure, sediment permeability measurement is performed by specifically introducing or injecting liquids, preferably water, into the underground to be examined by means of an injection member, respectively.

The term overpressure, in the sense of the disclosure, refers to the state at the exit port or the exit ports. Some overpressure is provided to guide the fluid from the inside to the outside through the exit ports. The height of the pressure level especially depends on the underground, but also on the desired period of time of the injection. If introduction is done over a long period of time, minimum overpressure may be sufficient. With specific undergrounds, it may even be possible to introduce the fluid without overpressure, but also at ambient pressure. Generally, overpressure of 1 bar has been proven to be especially advantageous.

The injection member preferably is formed of a lance of a resistant material having maximum rigidity. This is especially reasonable, as the lance is required to be introduced into the soil or underground to be examined. The injection member or the lance, respectively, has a length that allows conveyance and introduction of the lance into the underground manually by one person only. For example, the lance may be between 0.5 m and 1.5 m of length, preferably about 1 m. Depending on the consistency of the underground, the injection member may be pushed in, driven-in or screwed-in or drilled-in, respectively. Accordingly, the lance may comprise other members that facilitate introduction into the soil. Among them, there is an auxiliary impact weight, which is fixedly attached to the lance, or an especially resistant tip at the free end. A relatively coarse external thread is also conceivable, enabling turning or screwing into the underground by the user.

In an especially preferred embodiment, it has been proven that the diameter of the lance should be as small as possible, for example, 0.5 cm to 3 cm. The reason resides in that, following measurement, the still remaining liquid exits the interior space of the lance through the exit ports. In the subsequent measurement, the actually empty interior space is required to be filled before the liquid may be passed on to the environment.

In order to facilitate introduction of the lance into especially resistant underground and not to damage the lance, respectively, said lance, in turn, may be jacketed by another resistant hollow body. In this way, quasi a double tube will be formed, wherein the liquid is passed through the inner tube. In a first embodiment, an end-side opening of the inner tube leads immediately into an end-side opening of the outer tube, preferably at the free end of the lance, i.e. in the region of the lance tip.

In a second embodiment, the liquid first passes through the exit ports of the inner tubes to the annular space between the two tubes, to subsequently exit through the exit ports of the outer tube. For this purpose, the inner tube may have one or more exit ports.

Advantageously, the injection member may comprise an operable and closable valve, preferably a tap to be manually operated, in the entrance region of the liquid towards the injection member. The valve is open during measurement, it may be closed after completion of measurement, so that the liquid may no longer flow through the exit ports. In the embodiment having two coaxial tubes and while making use of the annular spaces interposed therebetween, it is sufficient for the outer tube to be closable via one valve.

Volume or pressure measurements are done with an appropriate measuring apparatus, which preferably is arranged at the top end of the lance, i.e. to be arranged during use exterior of the water.

Alternatively, the injection member, for example, may also be formed by tubing, which is laid into a riverbed, resting there until, due to sedimentation, it will be found in the underground.

Independently of forming the injection member, for example, as a lance or tubing, the injection member according to the disclosure may also be embedded into the underground.

In its interior space, the injection member comprises a liquid-conductive cavity, wherein at the free end of the injection member at least one exit port is provided. Through the cavity, the liquid will be guide to the exit port and will be injected into the surrounding underground

A pressure-increasing device will thereby cause increase of the pressure level of the liquid to be injected. The pressure-increasing device may be formed by a simple manually driven pump, but an electrically or motor-driven pump is also conceivable.

The liquid to be injected may either be taken from the environment, for example, from a water, but, according to the disclosure, there may as well be provided a receptacle, in which the liquid is stored. This approach has the advantage that the liquid to be injected may meet specific requirements. In an especially advantageous embodiment, pure water will be used, which has no adverse effect on the environment, especially on the water and the ground water. When using a receptacle, said receptacle may, for example, be in communication with a pump, by means of which the pressure level within the receptacle may be increased to the desired level. By a valve, preferably a manual or magnetic valve, the liquid will be supplied to the injection member from the pressurized receptacle through a discharge line. Alternatively, a submersible pump or even a centrifugal pump may be provided within the receptacle.

In order to obtain reproducible results, a pressure regulator may preferably be provided for precisely setting the desired overpressure. In an especially advantageous embodiment, a manual pump is provided to generate overpressure, for example, to 2 bar in the liquid receptacle. It thus will be assured by the pressure regulator that in one measurement only the desired overpressure, for example 1 bar, is to be supplied to the interior of the injection member.

Advantageously, the receptacle is provided with straps, which, for example, allow for said receptacle to be carried on the back. This is especially advantageous when wading through waters, in particular, as this allows for electrically driven measuring apparatuses to be attached to the receptacle and thus to be safely carried above the water level.

Instead of having only one single exit port, there may advantageously be a plurality of exit ports distributed across the outer surface of the injection member and having standard diameters. Basically, the diameter is to be selected depending on the underground to be examined, for example, dimensions in the range of 1 mm to 5 mm, preferably 1 mm to 2 mm have been proven to be reasonable. Depending on the underground, diameters of less than 1 mm may be suitable.

The advantage of having a plurality of exit ports, which are distributed across the outer surface of the injection member, resides in that the risk of clogging of all exit ports by sand or gravel is reduced compared to having only one single exit port. Alternatively, however, one single exit port being present at the tip of the injection member may also be of advantage. This is especially due to the fact that while individual exit ports being clogged, the rate of fluid discharged from the remaining non-clogged exit ports increases. This is not true with only one single port. In this respect, it is useful to select an appropriate lance or a suitable injection member depending on the underground to be examined.

In order to assure a predetermined timing for liquid injection, a time-controlled valve may be provided according to the disclosure. Alternatively, the period of time for liquid injection may be measured by an external stopwatch.

When conducting the measurement according to the disclosure or the method according to the disclosure, respectively, three parameters are to be considered:
1.) the injection pressure required
2.) the time, where
3.) a specified amount of water is injected into the sediment

Two of the three parameters are held constant, while measuring the third one. Said parameter corresponds to the respective permeability of the sediments. Through calibration while using sediments having defined permeability, comparison to measurands introduced for sediment characterization, e.g. Kf-value in m/s, will be enabled. It may be reasonable for the injection member to be a lance, as the penetration depth of the lance will always be the same. For this reason, the lance, at its outer face, advantageously comprises a scaling or color indicator. For example, the free end region may be colored in a clearly visible manner, starting from the tip of the lance. When pounding into the ground, care must only be taken for the colored region to penetrate into the soil. As it is preferred for the lance to be also useful under water, coloring should be selected such that it may well be recognized through the water. Alternatively or additionally, a pattern may be used, if this improves visibility.

If the effect on the surrounding material of the underground is to be kept as low as possible, it is provided, according to the disclosure, for the injection member to be introduced as slow as possible into the underground. For example, a weight or a spring force may be applied thereto, and, for example, may slowly be forced into the underground by means of a tripod.

During measurement, injection pressure (p) and time (t) will advantageously be held constant, and the amount of water injected (V) will be measured. In an experimental setup, the following procedure including said values has been proven to be reasonable:
1.) in a water receptacle, a pressure of 1 bar is set, and
2.) during a time period of 5 seconds,
3.) water will be injected into the sediment, and this amount of water will be measured electronically.

With p=1 bar, this yields a flow rate (Q=ml/5 s and/or m³/s), which reflects the permeability.

The pressure, through which the liquid is introduced into the underground, may be significantly higher or lower, depending on the application. For example, measurements may result in reasonable and robust outcomes, where the pressure is significantly lower than 1 bar. Measurements conducted with lower pressure will result in lower effect on the underground surrounding the injection member.

The higher the introduced water volume, i.e. the flow rate, the more permeable is the sediment and the lower is the colmation. The working pressure and the injection time may be adapted if required.

Basically, measurement of a parameter other than the flow rate is also possible. For this, e.g., the injected water volume and another parameter (injection pressure or time) may be held constant, and either time or loss of pressure may be measured.

Additional sensors may be introduced into the sediment together with the injection member, e.g. for measuring the temperature, oxygen content, conductivity, localization of the measuring point (GPS) or another parameter. The sensors may also be secured at the injection member or may be integrated therein.

The mechanical compartments described in the above-described method may be supplemented or replaced, respectively, by electronical components. Digital acquisition and automated further processing is possible. It may also be made use of for building ground exploration or for permeability measurements of ground water conductors.

The above-described method basically is suitable to be employed in the marine surroundings (e.g. harbor basin and off shore). More measurements in still waters, such as lakes, ponds and pools, may be conducted. The underground permeability measuring apparatus and method are also suitable to be employed in permanent and temporal waters.

By stationary continuous operation of injection members in waters, long-term studies on the colmation may be performed. Use of data loggers that include automated communication of the measuring outcomes to a digital system, is possible.

In an especially advantageous embodiment, all members that require electrical power are designed such that they may be operated beyond 9V or 12 V or 24 V. This minimizes the size of batteries or accumulators, and especially allows power supply via solar cells, which is especially beneficial during long-term field measurements in nature.

Identification of colmation require the surrounding soil to become modified as little as possible. Especially, introduction of the injection member must not result in compressions of the surrounding material, as measuring data become changed. The underground permeability measuring apparatus according to the disclosure allows slow introduction of the injection member into the underground, thereby reducing effect on the surrounding material. The lance itself is directly inserted into the underground, thus avoiding drilling, such as required in other methods. The minimally invasive technique according to the disclosure essentially does not cause any environmental impairments.

In addition, examinations may be performed manually, i.e. measurements may be performed by one single person, who, for example, wades through river or creek beds and thereby taking measurements at different locations. In this respect, portability of the underground permeability measuring apparatus is of vital importance. This is also true as for appropriate examination or mapping of undergrounds numerous measurements are required to be performed, causing rearrangement times or a method including large-scale apparatuses not to effectively obtain results. With the underground permeability measuring apparatus according to the disclosure, many measurements may be performed in very short time directly by manually introducing the lance. For this, only one single person is required.

As already set forth, the disclosure is focused on the underground permeability measuring apparatus according to the disclosure to be portable and to be suitable for environment-friendly measurement of colmation in undergrounds of waters of any type. It is thus very essential that rapid and smooth measurement be enabled with the measuring apparatus. In this regard, it is also essential that the measuring apparatus be configured such that it may be employed in water or in water-saturated underground, respectively. The measurement is to be minimally invasive, and to impair the underground or the already existing colmation as little as possible.

### BRIEF DESCRIPTION OF THE DRAWINGS

The disclosure will be explained in detail by way of the figures. It is to be understood that they are only exemplary, especially the size ratios are not to scale. The figures only represent one working example of the disclosure, wherein:

FIG. 1: is a preferred working example of an underground permeability measuring apparatus according to the disclosure, and

FIG. 2: is one example of an injection member according to the disclosure.

### DETAILED DESCRIPTION OF THE DRAWINGS

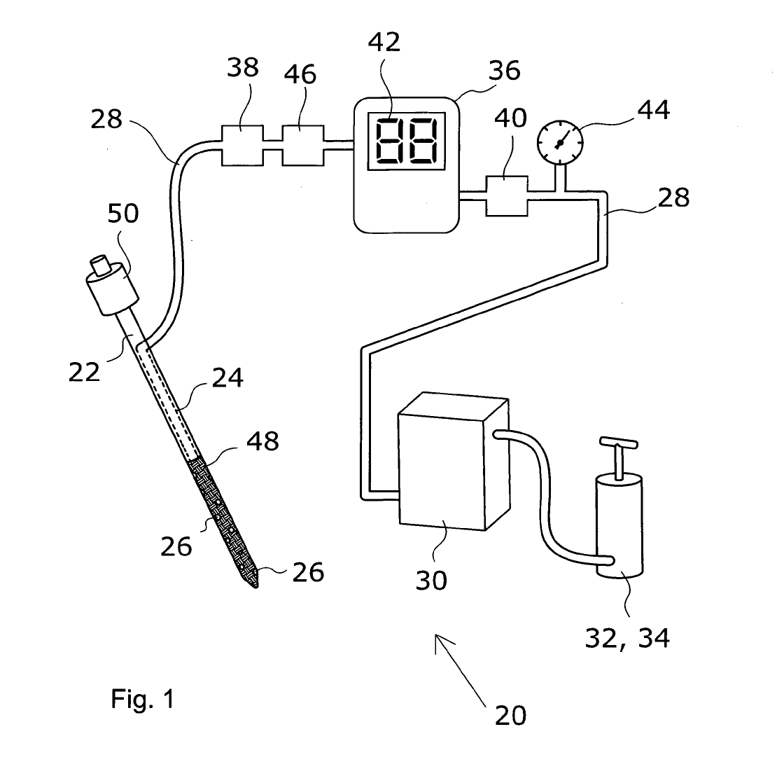

FIG. 1 shows an underground permeability measuring apparatus 20 for measuring permeability of an underground. It comprises an injection member 22, in the working example shown, it is designed as a lance, which is introducible into an underground.

A cavity 24 is formed in the interior of the injection member 22, through which cavity liquid may be guided to the exit ports 26. The exit ports 26 are disposed at the free end region and are equally distributed across the outer circumference of the injection member 22.

In the working example shown, the injection member 22 is coupled to a line 8, which finally terminates in a receptacle 30. Liquid, which is to be injected into the underground, is located within the receptacle 30.

A pressure-increasing device 32 is for increasing the pressure level, at which the liquid is injected into the underground. In the working example shown, the pressure-increasing device 32 is formed as a manual pump 34, through which the pressure level in the receptacle 30 may be increased.

The underground permeability measuring apparatus 20 furthermore comprises a measuring apparatus 36, by which a measurement may be performed, selected from the group consisting of injected liquid volume, timing of liquid injection or pressure level of liquid injection. Accordingly, members required for this, such as, for example, a volume measuring apparatus 38, a pressure measuring apparatus and/or a clock are available.

A time-controlled valve 40 opens the inlet into the injection member 22 for a predetermined time. This valve 40 may be formed as a manual valve or even a magnetic valve having time-setting feature.

A display device 42 is for displaying measured values. Via the display device 42, all relevant data may be displayed, for example the pressure level measured, the liquid volume injected or the timing of liquid injection. Especially, with the display device, the period of time to be measured may be set.

The display device 42 may be formed as an external or integral component of the measuring apparatus.

In the working example shown, an additional pressure display 44 is provided.

The predetermined overpressure or the desired pressure level, respectively, may be assured with the help of an adjustable overpressure valve 46.

It may be seen that the injection member 22 comprises a colored region 48, which indicates a desired penetration depth into the underground. This colored region may be omitted, but it may also be replaced by a scaling, for example, one displaying centimeters.

In order to facilitate introduction of the injection member 22 into the underground, an impact weight 50 is provided. Alternatively, a relatively large external thread is also conceivable, via which the injection member 22 may be screwed into the underground.

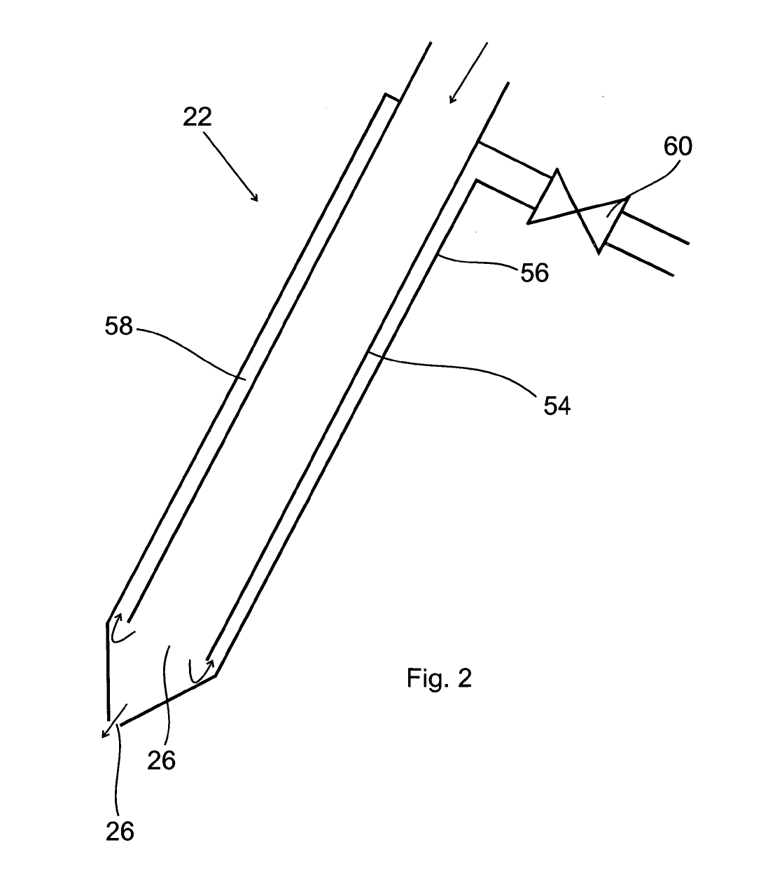

FIG. 2 demonstrates a working example of an injection member 22 having two coaxial tubes. The liquid flow is illustrated by arrows. An inner tube 54 is arranged in the interior of an outer tube 56. Through an exit port 26 of the inner tube 54 an annular space 58 of the injection member 22 is initially filled. Alternatively, to the single end side exit port 26 shown, the inner tube 54 may also comprise a plurality of exit ports 26. Following this, the liquid exits the outer tube 54 through the exit port 26. A valve 60, preferably a tap to be manually operated allows closure of the outer tube 54 or the annular space 58, respectively, thus avoiding unwanted leakage of the liquid through the exit port 26 after measurement.

The underground permeability measuring apparatus according to the disclosure and the method according to the disclosure firstly enable detection of colmation of running water sediments.

1. A portable underground permeability measuring apparatus for permeability measurement of soils, the apparatus comprising:
an injection member formed as a lance to be introduced into the underground, having a cavity for guiding a fluid to at least one exit port, which is arranged in the free end region of the injection member, through the injection member and the exit port into the underground surrounding the injection member is injectable.

2. The underground permeability measuring apparatus according to claim 1, wherein the pressure-increasing device is formed such that at least a predetermined pressure level is producible.

3. The underground permeability measuring apparatus according to claim 1, wherein at least one measuring apparatus is provided for at least one measurement selected from the group consisting of: fluid volume injected, timing of liquid injection, and pressure level of the fluid injection.

4. The underground permeability measuring apparatus according to claim 3, wherein a display device for displaying the measured values is provided.

5. The underground permeability measuring apparatus according to claim 1, wherein a receptacle for the fluid to be injected in fluid communication with the injection member is provided.

6. The underground permeability measuring apparatus according to claim 1, wherein the injection member comprises a plurality of exit ports distributed across the outer circumference of the injection members.

7. The underground permeability measuring apparatus according to claim 3, wherein a time-controlled valve is provided, by which timing of fluid injection is adjustable.

8. The underground permeability measuring apparatus according to claim 1, wherein the pressure-increasing device is formed by a manually operated pump.

9. The underground permeability measuring apparatus according to claim 1, wherein the pressure-increasing device is formed by an electrically operated pump.

10. A method for measuring colmation in undergrounds of waters, the method including the following steps:
introducing into an underground an injection member formed as a lance and having a cavity for guiding fluid to at least one exit port, which exit port is arranged in the free end region of the injection member,
injecting fluid by way of overpressure through an injection member and the exit port into the underground, while setting and maintaining two parameters, selected from the group consisting of:
a) fluid volume, injected,
b) timing of fluid injection, and
c) pressure level of fluid injection,
Identifying said parameter a), b) or c), which has not been set yet and maintained.

11. The method according to claim 10, wherein comparing the identified parameters to reference values for that parameter, the reference values having been previously identified for undergrounds having predetermined permeability.

12. The method according to claim 10, wherein the pressure level of the fluid injection is less than 1 bar.
